(19) United States
(12) Patent Application Publication
(10) Pub. No.: US 2004/0096455 A1
(43) Pub. Date: May 20, 2004

(54) Transdermal vaccine delivery device having coated microprotrusions

(76) Inventors: Yuh-Fun Maa, Millbrae, CA (US); Michel J.N. Cormier, Mountain View, CA (US); James Matriano, Mountain View, CA (US); WeiQi Lin, Palo Alto, CA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/637,909

(22) Filed: Aug. 8, 2003

Related U.S. Application Data
(60) Provisional application No. 60/402,269, filed on Aug. 8, 2002.

(51) Int. Cl. A61B 17/20; A61M 37/00; A61K 39/00

(52) U.S. Cl. 424/184.1; 604/46

(57) ABSTRACT

A device and method are provided for percutaneous transdermal delivery of a immunologically active agent. The agent is mixed with appropriate surfactants and dissolved in water to form an aqueous coating solution having the appropriate concentration for coating extremely tiny skin piercing elements. The coating solution is applied to the skin piercing elements using known coating techniques and then dried. The device is applied to the skin of a living animal, causing the microprotrusions to pierce the stratum corneum and deliver a immunologically effective dose of the immunologically active agent to the animal.

This application claims the benefit of U.S. Provisional Application No. 60/402,269, filed Aug. 8, 2002.

### TECHNICAL FIELD

This invention relates to administering and enhancing transdermal delivery of a vaccine across the skin. More particularly, the invention relates to a percutaneous vaccine delivery system for administering an immunologically active agent through the stratum corneum using skin piercing microprotrusions which have a dry coating of the immunologically active agent. The dry coating is formed from a solution containing the immunologically active agent and surfactants which has been applied to microprotrusions. Delivery of the agent is facilitated when the microprotrusions pierce the skin of a patient and the patient's interstitial fluid contacts and dissolves the immunologic agent.

### BACKGROUND

Drugs are most conventionally administered either orally or by injection. Unfortunately, many medicaments are completely ineffective or have radically reduced efficacy when orally administered since they either are not absorbed or are adversely affected before entering the bloodstream and thus do not possess the desired activity. On the other hand, the direct injection of the medicament into the bloodstream, while assuring no modification of the medicament during administration, is a difficult, inconvenient, painful and an uncomfortable procedure which sometimes results in poor patient compliance.

Vaccines, which are typically proteins molecules that form part of the membrane or outer coating of cells or viruses, are introduced into organisms in order to induce the production of antibodies to the organisms or viruses. Vaccines are typically weakened or killed viruses which are introduced into the body. This enables prevention of diseases in humans and animals.

Vaccines are traditionally administered through intramuscular oral, or subcutaneous injections. IV injections of vaccines are either not effective or practical. Transdermal delivery of vaccines is an alternative because of the immunological responsiveness of the skin.

Skin is not only a physical barrier that shields the body from external hazards, but is also an integral part of the immune system. The immune function of the skin arises from a collection of residential cellular and humoral constituents of the viable epidermis and dermis with both innate and acquired immune functions, collectively known as the skin immune system.

One of the most important components of the skin immune system are the Langerhan's cells (LC) which are specialized antigen presenting cells found in the viable epidermis. LC's form a semi-continuous network in the viable epidermis due to the extensive branching of their dendrites between the surrounding cells. The normal function of the LC's is to detect, capture and present antigens to evoke an immune response to invading pathogens. LC's perform his function by internalizing epicutaneous antigens, trafficking to regional skin-draining lymph nodes, and presenting processed antigens to T cells.

The effectiveness of the skin immune system is responsible for the success and safety of vaccination strategies that have been targeted to the skin. Vaccination with a live-attenuated smallpox vaccine by skin scarification has successfully led to global eradication of the deadly small pox disease. Intradermal injection using ⅕ to 1/10 of the standard IM doses of various vaccines has been effective in inducing immune responses with a number of vaccines while a low-dose rabies vaccine has been commercially licensed for intradermal application.

As an alternative, transdermal delivery provides for a method of administering vaccines that would otherwise need to be delivered via hypodermic injection or intravenous infusion. Transdermal vaccine delivery offers improvements in both of these areas. Transdermal delivery when compared to oral delivery avoids the harsh environment of the digestive tract, bypasses gastrointestinal drug metabolism, reduces first-pass effects, and avoids the possible deactivation by digestive and liver enzymes. Conversely, the digestive tract is not subjected to the vaccine during transdermal administration. However, in many instances, the rate of delivery or flux of many vaccines via the passive transdermal route is too limited to be immunologically effective.

The word “transdermal” is used herein as a generic term referring to passage of an agent across the skin layers. The word “transdermal” refers to delivery of an agent (e.g., a vaccine or a therapeutic agent such as a drug) through the skin to the local tissue or systemic circulatory system without substantial cutting or penetration of the skin, such as cutting with a surgical knife or piercing the skin with a hypodermic needle. Transdermal agent delivery includes delivery via passive diffusion as well as delivery based upon external energy sources including electricity (e.g., iontophoresis) and ultrasound (e.g., phonophoresis). While drugs do diffuse across both the stratum corneum and the epidermis, the rate of diffusion through the stratum corneum is often the limiting step particularly for larger proteins, peptides, oligonucleotides and vaccines. Many compounds, in order to achieve a immunologically effective dose, require higher delivery rates than can be achieved by simple passive transdermal diffusion. When compared to injections, transdermal agent delivery eliminates the associated pain and reduces the possibility of infection.

Transdermal drug delivery systems generally rely on passive diffusion to administer the drug while active transdermal drug delivery systems rely on an external energy source (e.g., electricity) to deliver the drug. Passive transdermal drug delivery systems are more common. Passive transdermal systems have a drug reservoir containing a high concentration of drug adapted to contact the skin where the drug diffuses through the skin and into the body tissues or bloodstream of a patient. The transdermal drug flux is dependent upon the condition of the skin, the size and physical/chemical properties of the drug molecule, and the concentration gradient across the skin. Because of the low permeability of the skin to many drugs, transdermal delivery has had limited applications. This low permeability is attributed primarily to the stratum corneum, the outermost skin layer which consists of flat, dead cells filled with keratin fibers (keratinocytes) surrounded by lipid bilayers. This highly-ordered structure of the lipid bilayers confers a relatively impermeable character to the stratum corneum.

One common method of increasing the passive transdermal diffusional drug flux involves pre-treating the skin with, or co-delivering with the drug, a skin permeation enhancer. A permeation enhancer, when applied to a body surface through which the drug is delivered, enhances the flux of the drug therethrough. However, the efficacy of these methods in enhancing transdermal protein flux has been limited, at least for the larger proteins, due to their size.

Active transport systems use an external energy source to assist drug flux through the stratum corneum. One such enhancement for transdermal drug delivery is referred to as “electrotransport.” This mechanism uses an electrical potential, which results in the application of electric current to aid in the transport of the agent through a body surface, such as skin. Other active transport systems use ultrasound (phonophoresis) and heat as the external energy source.

There also have been many attempts to mechanically penetrate or disrupt the outermost skin layers thereby creating pathways into the skin in order to enhance the amount of agent being transdermally delivered. Early vaccination devices known as scarifiers generally had a plurality of tines or needles which are applied to the skin to scratch or make small cuts in the area of application. The vaccine was applied either topically on the skin, such as U.S. Pat. No. 5,487,726 issued to Rabenau or as a wetted liquid applied to the scarifier tines such as U.S. Pat. No. 4,453,926 issued to Galy, or U.S. Pat. No. 4,109,655 issued to Chacornac, or U.S. Pat. No. 3,136,314 issued to Kravitz. Scarifiers have been suggested for intradermal vaccine delivery in part because only very small amounts of the vaccine need to be delivered into the skin to be effective in immunizing the patient. Further, the amount of vaccine delivered is not particularly critical since an excess amount also achieves satisfactory immunization. However a serious disadvantage in using a scarifier to deliver a vaccine is the difficulty in determining the transdermal dosage delivered. Also due to the elastic, deforming and resilient nature of skin to deflect and resist puncturing, the tiny piercing elements often do not uniformly penetrate the skin and/or are wiped free of a liquid coating of an agent upon skin penetration. Additionally, due to the self healing process of the skin, the punctures or slits made in the skin tend to close up after removal of the piercing elements from the stratum corneum. Thus, the elastic nature of the skin acts to remove the active agent coating which has been applied to the tiny piercing elements upon penetration of these elements into the skin. Furthermore the tiny slits formed by the piercing elements heal quickly after removal of the device, thus limiting the passage of agent through the passageways created by the piercing elements and in turn limiting the transdermal flux of such devices.

Other devices which use tiny skin piercing elements to enhance transdermal drug delivery are disclosed in European Patent EP 0 407063A1, U.S. Pat. No. 5,879,326 issued to Godshall, et al., U.S. Pat. No. 3,814,097 issued to Ganderton, et al., U.S. Pat. No. 5,279,544 issued to Gross, et al., U.S. Pat. No. 5,250,023 issued to Lee, et al., U.S. Pat. No. 3,964,482 issued to Gerstel, et al., Reissue 25,637 issued to Kravitz, et al., and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated by reference in their entirety. These devices use piercing elements of various shapes and sizes to pierce the outermost layer (i.e., the stratum corneum) of the skin. The piercing elements disclosed in these references generally extend perpendicularly from a thin, flat member, such as a pad or sheet. The piercing elements in some of these devices are extremely small, some having dimensions of only about 25-400 μm in length and a thickness of only about

5-50 μm. These tiny piercing/cutting elements make correspondingly small microslits/microcuts in the stratum corneum for enhanced transdermal agent delivery therethrough.

Generally, these systems include a reservoir for holding the drug and also a delivery system to transfer the drug from the reservoir through the stratum corneum, such as by hollow tines of the device itself. One example of such a device is disclosed in WO 93/17754 which has a liquid drug reservoir. The reservoir must be pressurized to force the liquid drug through the tiny tubular elements and into the skin. Disadvantages of devices such as these include the added complication and expense for adding a pressurizable liquid reservoir and complications due to the presence of a pressure-driven delivery system.

Instead of a physical reservoir, it is possible to have the drug that is to be delivered coated upon the microprojections. This eliminates the necessity of a reservoir and developing a drug formulation or composition specifically for the reservoir.

It is important when the agent solution is applied to the microprojections that the coating that is formed is homogeneous and evenly applied, preferably limited to the microprojections themselves. This enables greater dissolution of the agent in the interstitial fluid once the devices has been applied to the skin and the stratum corneum has been pierced, as compared to a coating distributed upon the whole array.

In addition, a homogeneous coating provides for greater mechanical stability both during storage and during insertion into the skin. Weak and discontinuous coatings are more likely to flake off during manufacture and storage and to be wiped off by the skin during application of the microprojections into the skin.

### DESCRIPTION OF THE INVENTION

The device and method of the present invention overcome these limitations by transdermally delivering an immunologically active agent using a microprotrusion device having microprotrusions which are coated with a dry homogeneous coating. This coating contains a sufficient amount of a surfactant which provides a coating containing an efficacious amount of vaccine and promotes the solubilization of the coating when introduced into the skin. The present invention is directed to a device and method for delivering an immunologically active agent through the stratum corneum of preferably a mammal and most preferably a human, by having a homogeneous coating on a plurality of stratum corneum-piercing microprotrusions.

These surfactants fall into several classes. There are those that are negatively charged such as SDS and the like. They can also be positively charged such as cetyl pyridinium chloride (CPC), TMAC, benzalkonium chloride or neutral, such as tween, sorbitan, or laureth.

Surfactants can be incorporated in the drug formulation used to coat the microprojections. A preferred embodiment of this invention consists of a device for delivering through the stratum corneum, a beneficial agent which has been coated on a plurality of microprotrusions by applying to the microprotrusions a solution of an immunologically active agent and a surfactant agent, which is then dried to form the coating. This coating solution preferabley contains from about 1 wt % to about 30 wt % surfactant. Optionally the microprotrusions are surface treated to enhance the uniformity of the coating that is formed on the microprotrusions. The device comprises a member having a plurality, and preferably a multiplicity, of stratum corneum-piercing microprotrusions. Each of the microprotrusions has a length of less than 600 μm, or if longer than 600 μm, then means are provided to ensure that the microprotrusions penetrate the skin to a depth of no more than 600 μm. These microprotrusions have a dry coating thereon. The coating, before drying, comprises an aqueous solution of a immunologically active agent and a surfactant. The immunologically active agent is applied to the microprojections as a solution which is sufficiently concentrated so that an immunologically effective dose can be applied to the microprojections. The amount is preferably in the range of about 1 microgram to about 500 micrograms. The solution, once coated onto the surfaces of the microprotrusions, provides an immunologically effective amount of the immunologically active agent. The coating is further dried onto the microprotrusions using drying methods known in the art.

Another preferred embodiment of this invention consists of a method of making a device for transdermally delivering an immunologically active agent. The method comprises providing a member having a plurality of stratum corneum-piercing microprotrusions. An aqueous solution of the immunologically active agent plus a surfactant is applied to the microprotrusions and then dried to form a dry agent-containing coating thereon. The immunologically active agent is sufficiently concentrated in the aqueous solution that an immunologically effective dose can be contained within the coatings. The composition can be prepared at any temperature as long as the immunologically active agent is not rendered inactive due to the conditions. The solution, once coated onto the surfaces of the microprotrusions, provides an immunologically effective amount of the immunologically active agent.

The coating thickness is preferably less than the thickness of the microprotrusions, more preferably the thickness is less than 50 μm and most preferably less than 25 μm. Generally, the coating thickness is an average thickness measured over the microprotrusions.

The most preferred agents are selected from the group consisting of conventional vaccines, recombinant protein vaccines, and therapeutic cancer vaccines.

The coating can be applied to the microprotrusions using known coating methods. For example, the microprotrusions can be immersed or partially immersed into an aqueous coating solution of the agent as described in pending U.S. application Ser. No. 10/099,604, filed Mar. 15, 2002. Alternatively the coating solution can be sprayed onto the microprotrusions. Preferably the spray has a droplet size of about 10-200 picoliters. More preferably the droplet size and placement is precisely controlled using printing techniques so that the coating solution is deposited directly onto the microprotrusions and not onto other “non-piercing” portions of the member having the microprotrusions.

In another aspect of the invention, the stratum corneum-piercing microprotrusions are formed from a sheet wherein the microprotrusions are formed by etching or punching the sheet and then the microprotrusions are folded or bent out of a plane of the sheet. While the pharmacologically active agent coating can be applied to the sheet before formation of the microprotrusions, preferably the coating is applied after the microprotrusions are cut or etched out but prior to being folded out of the plane of the sheet. More preferred is coating after the microprotrusions have been folded or bent from the plane of the sheet.

### BRIEF DESCRIPTION OF THE DRAWINGS

The invention will now be described in greater detail with reference to the preferred embodiments illustrated in the accompanying drawings and figures. wherein:

FIG. 1 is a perspective view of a portion of one example of a microprotrusion array;

FIG. 2 is a perspective view of the microprotrusion array of FIG. 1 with several types of coatings deposited onto the microprotrusions;

FIG. 3 is a perspective view of the microprotrusion array of FIG. 1 showing a pattern coating deposited onto the microprotrusions;

FIG. 4. is a graph showing effect of surfactant concentration on solubility of proteins and peptides.

FIG. 5 shows the chemical structure of a number of surfactants

FIG. 6 is a graph showing the in vivo immunological response by guinea pigs to HA that has been delivered to the test subject by means of a coated microprojection array.

### MODES FOR CARRYING OUT THE INVENTION

Definitions:

Unless stated otherwise the following terms used herein have the following meanings.

The term “transdermal” means the delivery of an agent into and/or through the skin for local or systemic therapy.

The term “transdermal flux” means the rate of transdermal delivery.

The term “co-delivering” as used herein means that a supplemental agent(s) is administered transdermally either before the agent is delivered, before and during transdermal flux of the agent, during transdermal flux of the agent, during and after transdermal flux of the agent, and/or after transdermal flux of the agent. Additionally, two or more beneficial agents may be coated onto the microprotrusions resulting in co-delivery of the beneficial agents.

The term “immunologically active agent” as used herein refers to a composition of matter or mixture containing a vaccine or other immunologically active agent which is immunologically effective when administered in a immunologically effective amount

The term “immunologically effective amount” or “immunologically effective rate” refers to the amount or rate of the immunologically active agent needed to stimulate or initiate the desired immunologic, often beneficial result. The amount of agent employed in the coatings will be that amount necessary to deliver an amount of the agent needed to achieve the desired immunological result. In practice, this will vary widely depending upon the particular immunologically active agent being delivered, the site of delivery, and the dissolution and release kinetics for delivery of the agent from the coating into skin tissues.

The term “microprotrusions” or “microprojections” refers to piercing elements which are adapted to pierce or cut through the stratum corneum into the underlaying epidermis layer, or epidermis and dermis layers, of the skin of a living animal, particularly a mammal and more particularly a human. The piercing elements should not pierce the skin to a depth which causes significant bleeding. Typically the piercing elements have a length of less than 500 microns, and preferably less than 250 microns. The microprotrusions typically have a width and thickness of about 5 to 50 microns. The microprotrusions may be formed in different shapes, such as needles, hollow needles, blades, pins, punches, and combinations thereof.

The term “microprotrusion array” or “microprotrusion member” as used herein refers to a plurality of microprotrusions arranged in an array for piercing the stratum corneum. The microprotrusion array may be formed by etching or punching a plurality of microprotrusions from a thin sheet and folding or bending the microprotrusions out of the plane of the sheet to form a configuration such as that shown in FIG. 1. The microprotrusion array may also be formed in other known manners, such as by forming one or more strips having microprotrusions along an edge of each of the strip(s) as disclosed in Zuck, U.S. Pat. No. 6,050,988. The microprotrusion array may include hollow needles which hold a dry pharmacologically active agent.

References to the area of the sheet or member and reference to some property per area of the sheet or member, are referring to the area bounded by the outer circumference or border of the sheet.

The term “pattern coating” refers to coating an agent onto selected areas of the microprotrusions. More than one immunologically active agent may be pattern coated onto a single microprotrusion array. Pattern coatings can be applied to the microprotrusions using known micro-fluid dispensing techniques such as micropipeting and ink jet coating. Tip coating, which refers to applying the coating on the very end of the microprotrusion, is the preferred type of pattern coating.

The term “solution” shall include not only compositions of fully dissolved components but also suspensions of protein virus particles, inactive viruses, and split-virions.

### DETAILED DESCRIPTION

The present invention provides a device for transdermally delivering an immunologically active agent to a patient in need thereof. The device has a plurality of stratum corneum-piercing microprotrusions extending therefrom. The microprotrusions are adapted to pierce through the stratum corneum into the underlying epidermis layer or dermis layers, but do not penetrate so deep as to reach the capillary beds and cause significant bleeding. The microprotrusions have a dry coating thereon which contains the immunologically active agent. Upon piercing the stratum corneum layer of the skin, the agent-containing coating is dissolved by body fluid (intracellular fluids and extracellular fluids such as interstitial fluid) and released into the skin.

The kinetics of the agent-containing coating dissolution and release will depend on many factors including the nature of the immunologically active agent, the coating process, the coating thickness and the coating composition (e.g., the presence of coating formulation additives). Depending on the release kinetics profile, it may be necessary to maintain the coated microprotrusions in piercing relation with the skin for extended periods of time (e.g., up to about 8 hours). This can be accomplished by anchoring the microprotrusion member to the skin using adhesives or by using anchored microprotrusions such as described in WO 97/48440, incorporated by reference in its entirety.

FIG. 1 illustrates one embodiment of a stratum corneum-piercing Microprotrusion Member 5 for use with the present invention. FIG. 1 shows a portion of the Member 5 member having a plurality of Microprotrusions 10. The Microprotrusions 10 extend at substantially a 90° angle from Sheet 12 having Openings 14. Sheet 12 may be incorporated into a delivery patch including a backing for Sheet 12 and may additionally include adhesive for adhering the patch to the skin. In this embodiment the microprotrusions are formed by etching or punching a plurality of Microprotrusions 10 from a thin metal Sheet 12 and bending Microprotrusions 10 out of the plane of the sheet. Metals such as stainless steel and titanium are preferred. Metal microprotrusion members are disclosed in Trautman et al, U.S. Pat. No. 6,083,196; Zuck U.S. Pat. No. 6,050,988; and Daddona et al., U.S. Pat. No. 6,091,975; the disclosures of which are incorporated herein by reference. Other microprotrusion members that can be used with the present invention are formed by etching silicon using silicon chip etching techniques or by molding plastic using etched micro-molds. Silicon and plastic microprotrusion members are disclosed in Godshall et al., U.S. Pat. No. 5,879,326, the disclosures of which are incorporated herein by reference.

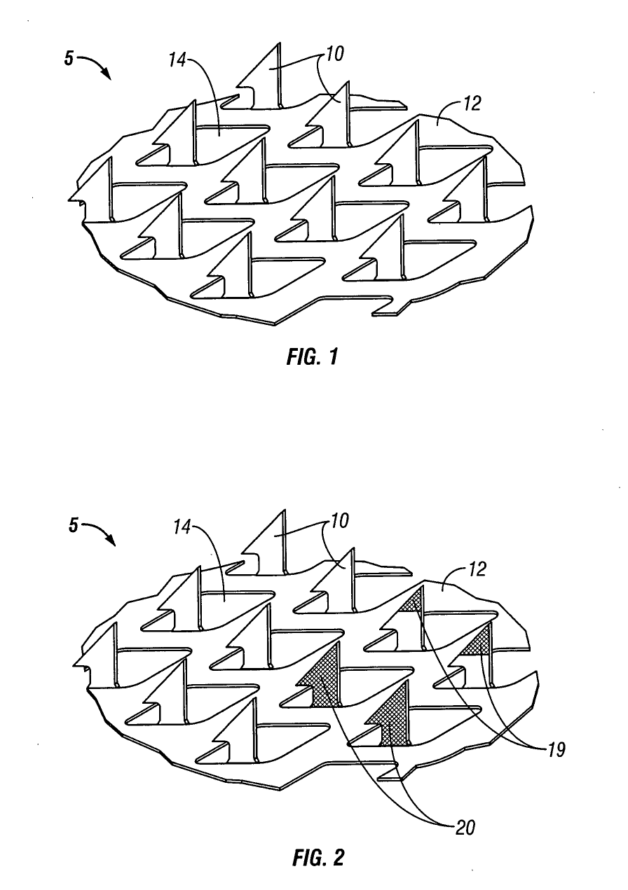

FIG. 2 illustrates the Microprotrusion Member 5 having a plurality of Microprotrusions 10, some of which have an immunologically active agent-containing Coating 16 or 20. These coatings may partially (Coating 19) or completely (Coating 20) cover the Microprotrusion 10. The coatings are typically applied after the microprotrusions are formed.

The coating on the microprotrusions can be formed by a variety of known methods. One such method is dip-coating. Dip-coating can be described as a means to coat the microprotrusions by partially or totally immersing the microprotrusions into the drug-containing coating solution. Alternatively the entire device can be immersed into the coating solution. Coating only those portions of the microprotrusion member which pierce the skin is preferred.

By use of the partial immersion technique described above, it is possible to limit the coating to only the tips of the microprotrusions. There is also a roller coating mechanism that limits the coating to the tips of the microprotrusion. This technique is described in a U.S. patent (Ser. No. 10/099,604 filed 16 Mar. 2002) which is fully incorporated herein by reference.

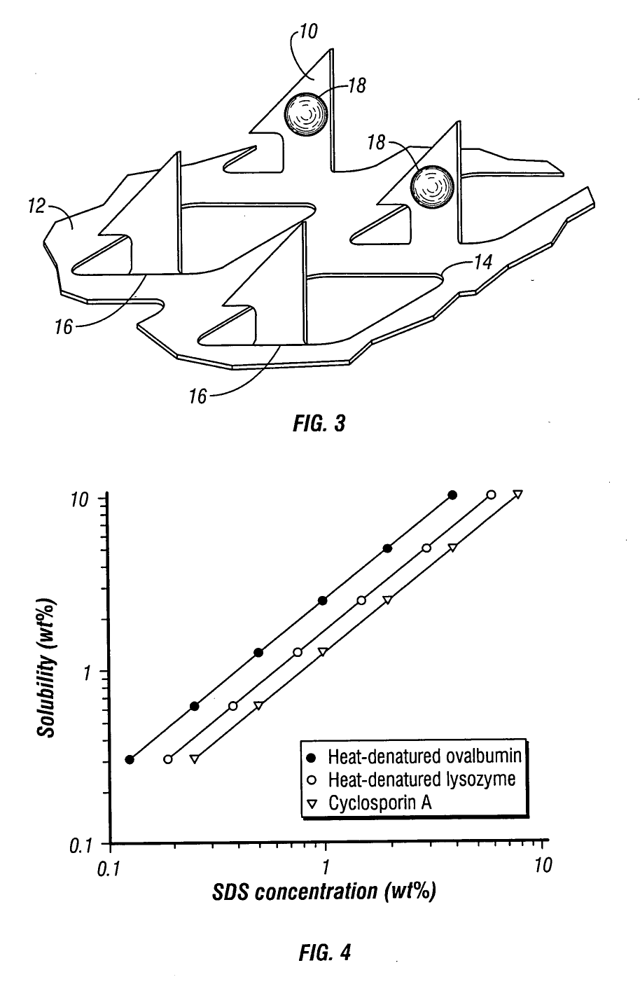

Other coating methods include spraying the coating solution onto the microprotrusions. Spraying can encompass formation of an aerosol suspension of the coating composition. In a preferred embodiment an aerosol suspension forming a droplet size of about 10 to 200 picoliters is sprayed onto the microprotrusions and then dried. In another embodiment, a very small quantity of the coating solution can be deposited onto the Microprotrusions 10 as shown in FIG. 3 as Pattern Coating 18. The Pattern Coating 18 can be applied using a dispensing system for positioning the deposited liquid onto the microprotrusion surface. The quantity of the deposited liquid is preferably in the range of 0.5 to 20 nanoliters/microprotrusion. Examples of suitable precision metered liquid dispensers are disclosed in U.S. Pat. Nos. 5,916,524; 5,743,960; 5,741,554; and 5,738,728 the disclosures of which are incorporated herein by reference. Microprotrusion coating solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

The desired coating thickness is dependent upon the density of the microprotrusions per unit area of the sheet and the viscosity and concentration of the coating composition as well as the coating method chosen. In general, coating thickness should be less than 50 microns since thicker coatings have a tendency to slough off the microprotrusions upon stratum corneum piercing. A preferred coating thickness is less than 25 microns as measured from the microprotrusion surface. Generally coating thickness is referred to as an average coating thickness measured over the coated microprotrusion.

The immunologically active agent used in the present invention requires a dose of about 1 micrograms to about 500 micrograms. Amounts within this range can be coated onto a microprotrusion array of the type shown in FIG. 1 wherein Sheet 12 has an area of up to 10 cm² and a microprotrusion density of up to 1000 microprotrusions per cm².

In all cases, after a coating has been applied, the coating solution is dried onto the microprotrusions by various means. In a preferred embodiment the coated device is dried in ambient room conditions. However, various temperatures and humidity levels can be used to dry the coating solution onto the microprotrusions. Additionally, the devices can be heated, lyophilized, vacuum dried or similar techniques used to remove the water from the coating.

Other known formulation adjuvants can be added to the coating solution as long as they do not adversely affect the necessary solubility and viscosity characteristics of the coating solution and the physical integrity of the dried coating. In addition, any additional formulation adjuvants should not significantly degrade the immunologically active agents immunogenic stimulating potency.

The following examples are given to enable those skilled in the art to more clearly understand and practice the present invention. They should not be considered as limiting the scope of the invention but merely as being illustrated as representative thereof.

Preliminary studies were performed to show the effectiveness of a surfactant in solubilizing proteins. The three proteins/peptides used in the first series of studies are ovalbumin (45 Kd), lysozyme (14 Kd) and the cyclosporin A (1.2 Kd).

A 10 wt % aqueous solution of each of the first two proteins were heat-denatured by exposing the solution to a temperature of 95° C. for 15 minutes. As a consequence of the denaturation, the two denatured proteins showed very low aqueous solubility. Cyclosporin A inherently exhibits low aqueous solubility.

Each of the three protein/peptide samples were used in the formulation of solutions having varying concentrations of SDS. The solubility of each sample, as expressed in terms of wt %, was measured and plotted against the concentration of SDS for that sample. This data is shown in FIG. 4.

It is clear that for the three test proteins, the solubility increased with increasing SDS concentration up to the highest concentration of SDS that was tested which was 10 wt %.

Other surfactants and concentrations were test against a solution 0.5 wt % ovalbumin. The data is given below in Table 1. Formulation that were effective in completely solubilizing the ovalbumin solution are indicated with a “+”, those that did not effect complete solubilization are marked with a “−”.

TABLE 1

| | Surfactant Concentration (M) | | | | |
|---|---|---|---|---|---|
| Surfactant | 0.0085 | 0.017 | 0.035 | 0.052 | 0.069 |
| Sodium octylsulfate | − | − | − | − | − |
| Sodium decylsulfate | − | − | − | + | + |
| Sodium dodecylsulfate | − | + | + | + | + |
| Sodium tetradecylsulfate | − | − | − | − | − |
| Sodium octadecylsulfate | − | − | − | − | − |
| Sodium laurate | − | − | + | + | + |
| Dodecyltrimethylammonium Br | − | − | − | − | − |
| Cetylpyridinium chloride | − | − | − | − | + |
| Tween 20 | − | − | − | − | − |
| Tween 80 | − | − | − | − | − |

A variety of surfactants have been evaluated in the influenza vaccine formulation for delivery via a microprotrusion array. A monovalent “split-varion” influenza vaccine (A/Panama/2007/99, H3N2) was used to evaluate various surfactants. To prepare this vaccine, influenza virus particles that are derived from egg embryos were split and extracted with surfactant and organic solvent according to standard protocols. After purification, the vaccine solution remains a suspension as it contains significant amounts of aggregated proteins and water-insoluble lipids.

A liquid formulation for microprotrusion array coating has to satisfy some liquid property criteria including sufficient solid content (vaccine content), liquid viscosity, favorable surface energy between the liquid formulation and the microprotrusion surface which is usually titanium. The “split-varion” flu vaccine preparation is a good material to use in the evaluation of the surfactants because the concentrated vaccine is highly turbid (milky white), which is probably the result of a suspension of split virus particles and aggregated proteins of various sizes. Using starting material of high turbidity makes it easier to evaluate the ability of the various surfactant formulations to solubilize the virus particles.

It is important to control the solubilization process in order to facilitate good coatings on the microprotrusions. Particulates in the suspension, particularly large particles (>10 μm), might interfere with or even disrupt the coating process. The second issue is the possibility of reducing antigenicity/immunogenicity of the aggregated antigen protein, hemagglutinin (HA) or other immunologically stimulating epitopes, upon delivery into the epidermal layer in the skin, especially when the aggregated HA particles are unable to return to an immunologically active form in the presence of interstitial fluid.

The surfactants used in this example are:

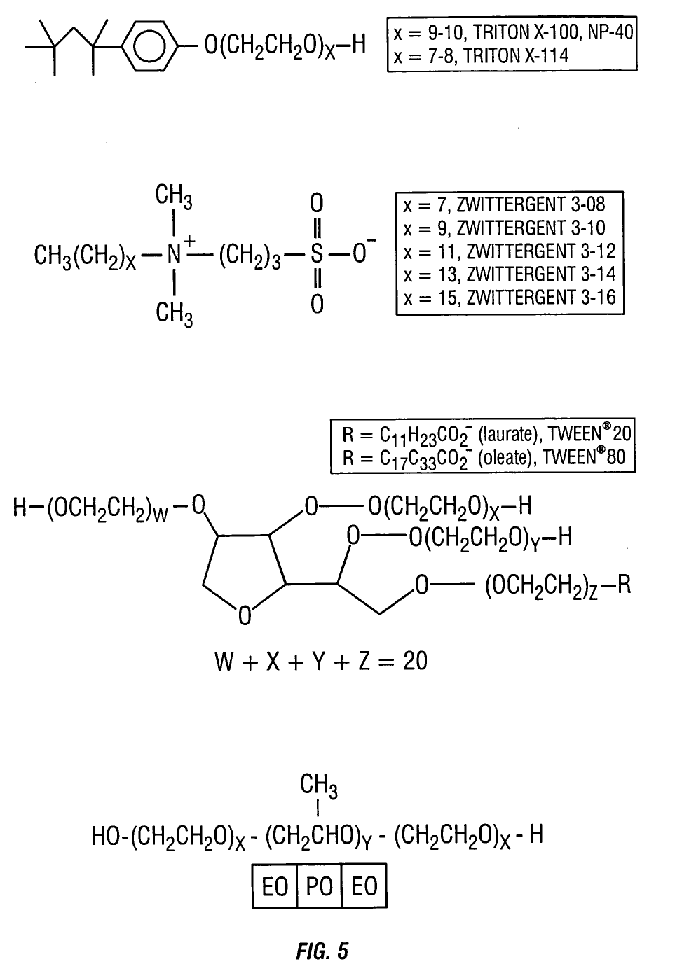

1. Triton X100 (see structure in the 1^{st} row in FIG. 5).

2. Zwittergent (see structure in the 2^{nd} row in FIG. 5).

3. Sodium dodecyl sulfate (SDS), CH₃(CH₂)₁₁SO₄⁻Na⁺.

4. Tween 20 or 80, polysorbate 20 or 80, (see structure in the 3^{rd} row in FIG. 5).

5. Pluronic F68, a block copolymer of propylene oxide (PO) and ethylene

oxide (EO). The propylene oxide block [PO] is sandwiched between

two ethylene oxide blocks [EO] (see structure in the 4^{th} row in FIG. 5.)

Surfactants 1-3 are strong surfactants which are known to denature the protein by actively binding the protein molecules to cause protein conformational changes. Therefore, despite their solubilizing ability, their tendency to denature proteins raises the concern about decreased antigenicity and immunogenicity of HA. Tween and Pluronic are milder compared to SDS, Triton, and Zwittergent so they might offer better long-term stability for the antigen.

Solubilizing Ability of Various Surfactants

The turbidity of the starting vaccine material was determined using UV/Visible spectrophotometry to determine the absorbance at 340 nm. The starting material, having an HA concentration of 80 μg/mL, was quite opalescent (see Table 2 where higher levels of absorbance are indicative of higher degrees of turbidity). After adjusting the solutions to bring them to a surfactant concentration of 0.1%, the vaccine solution clarified to different levels, suggesting that the solubilizing power of these surfactant follows the order of:

SDS≈Zwittergent 3-14>Triton X100>Tween 20≈Pluronic F68.

TABLE 2

| | | | 0.1% | 0.1% | 0.1% | 0.1% |
|---|---|---|---|---|---|---|
| | Starting | 0.1% | Triton | Zwittergent | Tween | Pluronic |
| | Material | SDS | X100 | 3-14 | 20 | F68 |
| Turbidity @ | 0.279 | 0.022 | 0.053 | 0.025 | 0.185 | 0.175 |
| 340 | | | | | | |
| nanometers | | | | | | |
| (80 μg/ml) | | | | | | |

Zwittergents were also evaluated. Zwittergents are a family of surfactants that are available with different hydrophobicities based on the number of methylene groups in the molecules (FIG. 5). Table 3 summarizes the solubilizing power of several different formulations containing 1 wt % of the indicated Zwittergent. Zwittergents with increasing hydrophobicity demonstrated increased solubilizing power as determined by turbidity measurements at 340 nanometers.

TABLE 3

| | Increasing Hydrophobicity | | | |
|---|---|---|---|---|
| | Increasing Solubilizing Power → | | | |
| | Starting | | | |
| | vaccine | | | |
| | material | | | |
| | (no added | Zwittergent | Zwittergent | Zwittergent |
| | surfactant) | 3-10 | 3-12 | 3-14 |
| Turbidity @ | 0.3557 | 0.120 | 0.087 | 0.070 |
| 340 | | | | |
| nanometers | | | | |
| (200 μg/ml) | | | | |

Pre-Formulation Process Evaluation

Commercial vaccine preparations typically contain HA from at least three different influenza strains. The starting vaccine material described herein contains only a single type and strain (A/Panama). This material has an HA concentration of 0.4 mg/mL.

As influenza virus is grown on chicken eggs, the starting material formulations contain not only the HA but other material such as proteins and lipids from the eggs that has not been removed. Because many patients are allergic to eggs and to reduce the exposure of the patients to other possibly sensitizing material, it is necessary to remove as much as possible, the non-HA material that is in the starting material.

In view of the above, the starting vaccine material will be buffer exchanged and highly concentrated. The following procedures were performed to the starting vaccine material as a prerequisite for preparing coating formulations:

### Diafiltration/Concentration by Tangential Flow Filtration (TFF)

Diafiltration was performed against water for injection (WFI). In the TFF system, 500 mL of starting vaccine material was concentrated to 50 mL in the TFF apparatus, which was then diafiltered with 2×500 mL of the diafiltration solution and then concentrated to a final volume having an HA concentration of approximately 10 mg/mL.

### Freeze-Drying

The solution above was freeze-dried in the presence of a sugar, either sucrose or a trehalose dihydrate. The chemical composition of the freeze dried material is summarized in Table 4:

TABLE 4
Chemical composition of the freeze dried vaccine

| Component | Composition |
|---|---|
| HA | 44.1% |
| Trehalose | 9.2% |
| Non-HA materials | 41.4% |
| 2-phenoxyethanol | 5.3% |

### Reconstitution with a Surfactant-Containing Liquid Formulation

The ability of the four solutions shown in Table 5 (below), to reconstitute the freeze dried material were evaluated as part of the overall determination of the proper reconstitution solution needed in order to provide a formulation with an HA concentration of 50 mg/ml.

TABLE 5

| | HA | | |
|---|---|---|---|
| | Concentration | HA/Surfactant | |
| Reconstitution | (mg/mL) | (w/w) | Comment |
| 8% SDS 70 μL | 37 | 1.0/2 | Nearly clear |
| | | | solution |
| 10% Triton 100 μL | 28 | 1.0/3.6 | Clear |
| | | | solution |
| 5% | 34 | 1.0/1.5 | Clear |
| Triton/Na₂CO₃— | | | solution |
| NaHCO₃ pH 10, | | | |
| 80 μL | | | |
| 6% Zwittergent | 38 | 1.0/1.3 | Slightly turbid |
| 2-14 70 μL | | | solution |

Based upon the evaluation of the various reconstitution formulations shown in Table 5, further studies were performed and the following formulations were effective in reconstituting the freeze dried HA solutions to an HA concentration of 50 mg/ml.

| Surfactant | Clarity of solution |
|---|---|
| 10% Zwittergent 3-14 | semi-clear |
| 5% Zwittergent 3-14/pH 10 buffer (sodium | semi-clear |
| carbonate/bicarbonate) | |
| 10% Triton X100/pH 10 | semi-clear |
| 10% SDS | semi-clear |
| 2% Tween 80/5% sucrose | turbid |
| 2% Pluronic F68/2.5% trehalose/2.5% mannitol | turbid |

After drying, the composition of each component of three of the above formulation could be estimated as shown below in Table 6.

TABLE 6:
Per cent Composition of the Three
10%-surfactant reconstituted formulations.

| | | 10% Triton | |
|---|---|---|---|
| Component | 10% Zwittergent 3-14 | X100 | 10% SDS |
| HA | 24.0% | 24.5% | 23.9% |
| Trehalose | 4.8% | 4.9% | 4.9% |
| Non-HA | 21.2% | 21.7% | 21.5% |
| materials | | | |
| Surfactant | 50.0% | 49.0% | 50.7% |
| Buffers | No | Negligible | No |
| Total | 100% | 100% | 100% |

The surfactant is the major component of each formulation, comprising of approximately 50% of the total solid.

### Liquid Properties (Viscosity, Contact Angle, Solid Content)

Liquid formulation parameters critical to microprotrusion coating were determined for various formulations prior to coating. These parameters, which include viscosity, wettability, and the solid content are given in Table 7.

The contact angle is measured by placing a known volume of the formulation on the surface to a 1 cm² titanium disc. The contact angle can be defined as the angle between the substrate support surface and the tangent line at the point of contact of the liquid droplet with the substrate.

Compared to pure water which has a contact angle of 73°, or non-surfactant formulations, the presence of a surfactant in a formulation improves wettability of the liquid formulation onto the titanium surface as evidenced by the decrease in the contact angle. Microprotrusion coating was performed to understand how these surfactants affect coating performance.

TABLE 7

| | Contact | Viscosity @200 | Solid |
|---|---|---|---|
| Formulation | angle | rpm (poise) | content (%) |
| 10% Zwittergent | 30° | 0.09 | 20 |
| 3-14 | | | |
| 5% Zwittergent 3-14 | 32° | 0.14 | 15 |
| at pH 10 | | | |
| 10% Triton X100 | 40° | 0.44 | 20 |
| at pH 10 | | | |
| 10% SDS | 30° | 0.21 | 20 |
| 2% Tween/ | 38° | 0.41 | 17 |
| 5% sucrose | | | |
| 2% Pluronic/ | 44° | NA | 17 |
| 2.5% trehalose/ | | | |
| 2.5% mannitol | | | |

Coating Feasibility

A 250-μL coater was used for all coating experiments. This coater is equipped with water input lines which allow addition of fresh water by a syringe pump to compensate water loss/evaporation during coating. The rate of water addition is 3-μL/minute. The linear coating speed is 1.15 cm/s. The arrays have a 2 cm² surface area. We applied 12 coats in all formulations/designs

All coatings show acceptable coating morphology based upon examination by SEM. It appears that these surfactants promote tip-coating, i.e. the position of the coating being close to the tip of the microprojection. Such coating location is considered preferable as coating too far away from the tip might be undeliverable if penetration doesn't carry that portion of coating far enough into the skin to be dissolved by interstitial fluid. This tip-coating is difficult to control with formulations either lacking surfactants or in the presence of insufficient amount of these surfactants.

### Delivery Results

Further studies were performed in order to determine the efficiency of delivery into the skin of HA from microprotrusions that were dry coated with various HA formulations. The delivery study was performed on hairless guinea pigs. A series of microprotrusion arrays were coated with the formulations shown in Table 8 below. The formulations also contained Fluorescein, a fluorescent marker.

After the application of a coated microprotrusion array to the skin for a predetermined period of time, Fluorescein determinations were made from samples collected from three sources. The first was a determination of the Fluorescein in skin biopsies taken from the microprotrusion array application site. The application period was short enough that Fluorescein delivered to the skin did not have time to migrate beyond that area of skin that was biopsied. The second source was from undissolved residue found on the microprotrusion array. The third was from a solution used to rinse off surface material found at the skin application site immediately after removal of the microprotrusion array.

Delivery efficiency is defined as the percentage Fluorescein in the skin relative to total amount recovered. The delivery studies were performed and the results are summarized in Table 8.

TABLE 8

| Coated Formulation | Delivery (%) |
|---|---|
| 10% Zwittergent 3-14 | 55.5 |
| 5% Triton/pH 10 | 60.4 |
| 10% SDS | 52.1 |
| 5% HA/5% sucrose/ | 45.1 |
| 2% Tween 80 | |
| 5% HA/2% pluronic/ | 73.2 |
| 2.5% trehalose/ | |
| 2.5% mannitol | |
| 5% HA/5% sucrose/ | 60.3 |
| 2% Tween 80 | |

All formulations/delivery conditions showed good delivery efficiency of >45%. This level of delivery efficiency might be attributed to the preferable coating positioning (tip coating) which allows most of the coating to penetrated well into the skin. These results confirm an important attribute of these surfactants, which not only facilitate solubilization of the flu vaccine but also the ability to modify the liquid properties of the coating formulations to promote effective tip coating. Within the range of effective penetration, tip coating improves delivery efficiency. The minimum delivery efficiency which would still provide sufficient amount of the immunologically active agent is considered to be 10%.

### HA Potency Assays

In addition to acceptable levels of delivery of the HA, it must also be shown that the HA that is delivered is still antigenic despite the treatment with the various surfactants. Two tests are used to measure the antigenicity of an HA formulation after treatment with the various surfactant formulations. These tests are a proprietary ELISA determination and a Western Blot.

### ELISA

The HA formulations were prepared as described above resulting in several surfactant formulations, both in the liquid and the dry states. ELISA determinations were performed on these samples. The results are summarized in Table 9. The HA content was determined by the bicinchoninic acid (BCA) total protein assay. Results from the BCA assay are consistent with the target HA concentration (0.4 mg/mL). Significant variations were seen in the SDS-containing formulation between several repeated assays. Because an ELISA assay depends in large part on the ability of the added antibody to bind to the antigen in the tested sample, overall, the ELISA results indicate that the HA in these surfactant formulations remains antigenic.

Sample 1 is the original HA material processed as described above. Samples 2-liquid through 5-liquid are replicates of the sample 1 which have been reconstituted in one of the four formulations indicated in the second column. Samples 2-solid through 5-solid are duplicates of samples 2-liquid through 5-liquid which have been air dried on 1 cm² titanium discs and then reconstituted in water. Samples 2-solid through 5-solid are meant to simulate the conditions of a coating on a titanium microprotrusion. The total protein for samples 2-solid through 5-solid were below the detectability threshold for the BCA assay.

TABLE 9

| | | HA by | ELISA |
|---|---|---|---|
| Sample # | Formulation | BCA (μg) | (%) |
| 1 | Freeze dried (FD) | 0.391 ± 0.012 | 78.8 |
| 2-liquid | FD/reconstituted with | 0.385 ± 0.010 | 96.7 |
| | 10% Zwittergent 3-14 | | |
| 3-liquid | FD/reconstituted with | 0.389 ± 0.005 | 79.8 |
| | 5% Zwittergent 3-14/ | | |
| | pH 10 | | |
| 4-liquid | FD/reconstituted with | 0.380 ± 0.008 | 94.5 |
| | 10% Triton X100/pH | | |
| | 10 | | |
| 5-liquid | FD/reconstituted with | 0.383 ± 0.009 | 231.0 |
| | 10% SDS | | |
| 2-solid | FD/reconstituted with | — | 97.7 |
| | 10% Zwittergent 3-14 | | |
| 3-solid | FD/reconstituted with | — | 71.4 |
| | 5% Zwittergent 3-14/ | | |
| | pH 10 | | |
| 4-solid | FD/reconstituted with | — | 91.6 |
| | 10% Triton X100/pH | | |
| | 10 | | |
| 5-solid | FD/reconstituted with | — | 37.0 |
| | 10% SDS | | |

### Western Blot

Sheep anti-HA antibodies were tested against 5 formulations of HA (the first five samples shown in Table 9 above). The samples were run on SDS-PAGE gels and stained with Commassie Blue. Molecular weight markers and the starting vaccine material were run along with the 5 formulations. The banding pattern for each of the 5 samples were very similar to that of the starting vaccine material indicating that there was no significant alteration in the samples as a consequence of being exposed to the surfactant formulations.

After a Western Blot was performed on the PAGE-gel, no differences were noticed among different formulations. A series of bands, reflecting the binding between proteins and the sheep anti-HA antibodies occurred primarily at high molecular weighs. There were three bands having an estimate molecular weight of approximately 75 kD, 150 kD and 225 kD which are presumed to be HA monomer, dimer, and trimer. Therefore, based on the matched bands and band intensity (relative to the starting vaccine), we would conclude that antigen HA in formulations that had been freeze-dried and exposed to a high concentration of a strong surfactant maintains its antigenicity.

Both ELISA and Western Blot analysis shows that HA maintains its antigenicity in the presence of these surfactants. However, the preservation of immunogenicity needs to be demonstrated.

### In Vivo Immunization Study

The final test is to determine the in vivo immunogenicity of preparations of HA which contain the various surfactants of interest. The formulations are given below in Table 10.

Each group tested consisted of 5 animals and each were given a primary vaccination on day 0 and a boost vaccination on day 28. The antigen dose in each case was 5 μg of HA as determined by BCA assay and delivered by IM injection. Sera was collected on day 28, 35 and 42.

TABLE 10
Immunization Formulations

| Group | Tested Formulation |
|---|---|
| 1 | HA (starting material) 0.401 mg/ml |
| 2 | 50 mg/ml HA 10% Zwittergent 3-14 |
| 3 | Same as Group 2, but dry-coated on |
| | titanium then reconstituted in sterile |
| | saline |
| 4 | 50 mg/ml HA 5% Zwittergent 3-14 |
| | pH 10 |
| 5 | Same as Group 4, but dry-coated on |
| | titanium then reconstituted in sterile |
| | saline |
| 6 | 50 mg/ml HA 10% Triton x100 |
| | pH 10 |
| 7 | Same as Group 6, but dry-coated on |
| | titanium then reconstituted in sterile |
| | saline |
| 8 | 50 mg/ml HA 10% SDS |
| 9 | Same as Group 8, but dry-coated on |
| | titanium then reconstituted in sterile |
| | saline |

Once the HA was concentrated and in the presence of surfactants, 5 μL (i.e., 200-260 μg HA) of the solution was aliquoted into a sterile tube (i.e., “liquid”). Another 5 μL was aliquoted onto a 1 cm² titanium disk and air dried (i.e., “dry-coated”). Both the “liquid” and “dry-coated” preparations were stored at −80° C. To determine the HA content by ELISA, the samples were thawed and reconstituted in 1 mL sterile saline. 0.5 mL of this material was used for the ELISA assay. The remaining 0.5 mL solution was stored at −80° C. On the day of the scheduled immunization date the remaining 0.5 ml sample was thawed and reconstituted in sterile saline to a concentration of 0.05 mg HA/mL.

Based on the data generated from the BCA assay, the 0.5 mL solution should contain 100-130 μg HA that was prepared from each formulation. The HA content measured by ELISA for all formulations (primary [d0] and booster [d28] preparations) can be seen in Table 11. As can be seen (last two columns), the HA activity measured by ELISA is generally lower than the estimates based on the BCA assay (exception d0 group 8). Of course, the BCA assay measures total protein content; thus an indirect measurement for HA. Because the ELISA has not been completely validated as an assay for HA quantification, we choose to use the BCA data to determine the volume of saline needed to dilute the HA to 50 μg/mL. Once formulations were diluted, 5 μg HA (0.1 mL) of each preparation was injected intramuscularly into each HGP (Table 10).

TABLE 11

| | | | HA dose | HA dose | |
|---|---|---|---|---|---|
| | | | calculated | calculated by | |
| | | | by BCA | ELISA (μg) | |
| Treatment | | HA | total protein | Prime | Boost |
| Group | HA formulation^{a} | state | assay (μg) | (d 0) | (d 28) |
| 1 | Starting Material | Liquid | 5 | NA | NA |
| 2 | 10% Zwittergent | Liquid | 5 | 3.06 | 3.59 |
| | (3-14) | | | | |
| 3 | 10% Zwittergent | Dry- | 5 | 2.85 | 4.98 |
| | (3-14) | coated | | | |
| 4 | 5% Zwittergent | Liquid | 5 | 2.70 | 4.50 |
| | (3-14) pH 10 | | | | |
| 5 | 5% Zwittergent | Dry- | 5 | 2.14 | 4.29 |
| | (3-14) pH 10 | coated | | | |
| 6 | 10% Triton X-100 | Liquid | 5 | 2.66 | 2.89 |
| 7 | 10% Triton X-100 | Dry- | 5 | 2.01 | 3.57 |
| | | coated | | | |
| 8 | 10% SDS | Liquid | 5 | 9.98 | 1.85 |
| 9 | 10% SDS | Dry- | 5 | 1.08 | 2.33 |
| | | coated | | | |

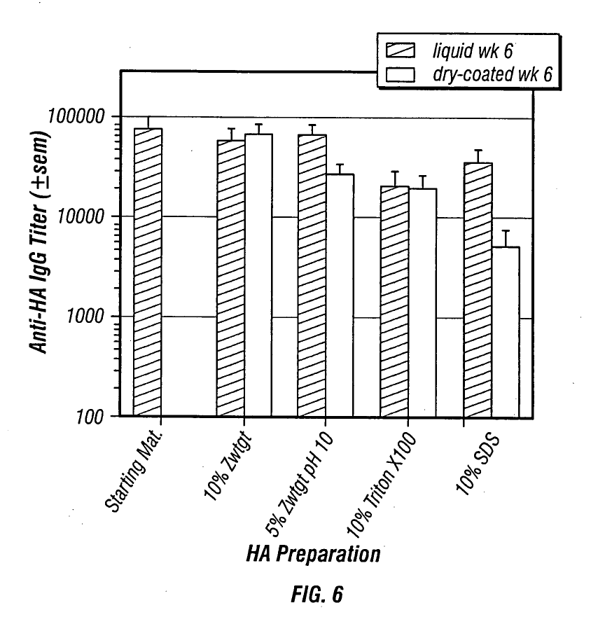

The average anti-HA titers from each treatment group were calculated and are shown FIG. 6 (d 42; 14 days after the booster injection).

The material which was reconstituted from liquid is shown as solid bars and the material which was dry coated on titanium discs and then reconstituted is shown as open bars.

Some preliminary statistical analysis was performed (individual titer values were log transformed). ANOVA showed no significance among starting material the four “liquid” formulations. However ANOVA did show significance among “dry-coated” formulations. The Least Significant Difference Test showed that the 10% SDS “dry-coated” formulation was statistically significant from:

| | |
|---|---|
| Starting Material | (p < 0.01); |
| 10% Zwittergent | (p < 0.01); |
| 5% Zwittergent, pH 10 | (p < 0.05); and |
| 10% Triton X-100 | (p < 0.05) |

The Least Significant Difference Test also showed that the 10% Zwittergent SDS “dry-coated” formulation (group 3) was statistically significant from 10% Triton X-100 (p<0.05). The t-Test (Grouped) analysis showed significance between “liquid” vs “dry-coated” formulation containing 10% SDS (group 8 versus group 9, p<0.05).

Overall, all surfactant-containing formulations, liquid or dry, remained immunogenic despite the exposure to the various surfactants. In addition, these formulations, with the exception of the SDS-containing formulation, elicited immune responses comparable to that by the starting vaccine. The lower immune responses shown by the SDS formulation might be due to the lower HA dose given as determined by the ELISA assay (Table 10).

Although the examples cited have formulations containing one surfactant, the invention should be understood to also include formulations containing two or more surfactants in combination.

Although the present invention has been described with reference to specific examples, it should be understood that various modifications and variations can be easily made by a person having ordinary skill in the art without departing from the spirit and scope of the invention. Accordingly, the foregoing disclosure should be interpreted as illustrative only and not to be interpreted in a limiting sense. The present invention is limited only by the scope of the following claims.

What is claimed is:

1. A device for transdermally delivering an immunologically active agent, the device comprising:
a member having a plurality of stratum corneum-piercing microprotrusions and
a dry coating on said member; said coating, before drying, comprising an aqueous solution of an amount of an immunologically active agent and a surfactant;
wherein said surfactant is present in the range of about 1 to about 30 wt % in said aqueous solution.

2. The device of claim 1 wherein said immunologically active agent is present in said aqueous solution in a concentration of at least about 1 wt %.

3. The device according to claim 2 wherein said coating is applied only to one or more of said microprotrusions.

4. The device according to claim 2 wherein the length of the microprotrusions is equal to or less than about 600 micrometers.

5. The device according to claim 2 wherein the total amount of said immunologically active agent coated on said member is between about 1 microgram and about 500 micrograms.

6. The device according to claim 2 wherein the thickness of said coating is equal to or less than about 50 micrometers.

7. The device according to claim 2 wherein the thickness of said coating is equal to or less than about 25 micrometers.

8. The device according to claim 2 wherein said immunologically active agent is selected from the group consisting of conventional vaccines, recombinant protein vaccines and therapeutic cancer vaccines.

9. The device according to claim 2 wherein said aqueous solution further comprises a suspension of one or more components selected from group consisting of protein virus particles, inactive viruses, and split-virions.

10. The device according to claim 2 wherein said member has an area of less than or equal to about 10 cm².

11. The device according to claim 2 wherein said member has a microprotrusion density of less than or equal to about 1000 microprotrusions per cm².

12. The device according to claim 2 wherein said immunologically active agent comprises hemagglutinin from at least one strain of influenza virus.

13. The device according to claim 2 wherein said surfactant is selected from the group consisting of sodium decylsulfate, sodium dodecylsulfate, sodium laurate, cetylpyridinium chloride, Zwittergent 3-10, Zwittergent 3-12, Zwittergent 3-14, Triton x-100, polysorbate 20, polysorbate 80 and Pluronic F68.

14. A transdermal drug delivery device comprising
a microprotrusion array have a plurality of microprotrusions; said microprotrusions being designed to pierce the stratum corneum when said microprotrusions array is applied to a body surface;
one or more of said microprotrusions being at least partially covered with an essentially dried coating containing at least one vaccine and at least one surfactant;
said coating containing a predetermined amount of said vaccine; wherein said predetermined amount is in the range of from about 1 microgram to about 500 micrograms of said vaccine;
said coating having been formed from a solution containing about 1 wt % to about 30 wt % of said surfactant;
said predetermined amount of said vaccine being sufficient to cause an immunological response when said vaccines is delivered transdermally; and
wherein the delivery efficiency of said immunologically active agent is greater than or equal to about 10%.

15. The device of claim 14 wherein said vaccine is present in said aqueous solution in a concentration of at least about 1 wt %.

16. The device according to claim 14 wherein said coating is applied only to one or more of said microprotrusions.

17. The device according to claim 14 wherein the length of the microprotrusions is equal to or less than 600 micrometers.

18. The device according to claim 14 wherein the thickness of said coating is equal to or less than about 50 micrometers.

19. The device according to claim 14 wherein the thickness of said coating is equal to or less than about 25 micrometers.

20. The device according to claim 14 wherein said vaccine is selected from the group consisting of conventional vaccines, recombinant protein vaccines and therapeutic cancer vaccines.

21. The device according to claim 14 wherein said aqueous solution further comprises a suspension of one or more components selected from group consisting of protein virus particles, inactive viruses, and split-virions.

22. The device according to claim 14 wherein said member has an area of less than or equal to about 10 cm².

23. The device according to claim 14 wherein said member has a microprotrusion density of less than or equal to about 1000 microprotrusions per cm².

24. The device according to claim 14 wherein said vaccine comprises hemagglutinin from at least one strain of influenza virus.

25. The device according to claim 14 wherein said surfactant is selected from the group consisting of sodium decylsulfate, sodium dodecylsulfate, sodium laurate, cetylpyridinium chloride, Zwittergent 3-10, Zwittergent 3-12, Zwittergent 3-14, Triton x-100, polysorbate 20, polysorbate 80 and Pluronic F68.
